(19) United States
(12) Patent Application Publication
(10) Pub. No.: US 2012/0310131 A1
(43) Pub. Date: Dec. 6, 2012

(54) PODIATRIC DEVICE

(76) Inventor: Uriel Rosen, Nesher (IL)

(21) Appl. No.: 13/559,609

(22) Filed: Jul. 27, 2012

Related U.S. Application Data
(63) Continuation-in-part of application No. PCT/IB2011/050363, filed on Jan. 27, 2011.

(30) Foreign Application Priority Data
Jan. 27, 2010 (GB) GB1001297.9

(51) Int. Cl. A61F 5/01

(52) U.S. Cl. 602/29

(57) ABSTRACT

A podiatric device useful for treating, preventing and alleviating the symptoms and discomfort caused by hallux valgus. The podiatric device includes a foothold with a first hinge forming element and an anterior part with a second hinge forming element. The first and second hinge forming elements forming a hinge allowing angular translation of the anterior part relatively the foothold, preferably a controllable pivoting movement.

### CROSS REFERENCE TO RELATED APPLICATIONS

This application is a continuation in part of PCT/IB2011/050363, entitled “Podiatric Device” and filed on Jan. 27, 2011 which in turn claims priority from patent application GB 1001297.9, now patent number GB2477281, entitled “Podiatric Device”, filed on Jan. 27, 2010; the entire contents of both of which are hereby incorporated by reference.

### FIELD OF THE INVENTION

The present invention generally relates orthopedic devices. In particular, the invention relates to a podiatric device useful for treating hallux valgus or bunion deformity at the metatarsophalangeal joint of the hallux.

### BACKGROUND OF THE INVENTION

U.S. Pat. Nos. 4,644,940; 5,529,075; 5,843,085; 6,093,163; 6,318,373; 6,391,031; and 6,964,645 are believed to represent the current state-of-the-art in the pertinent technical field.

### SUMMARY OF THE INVENTION

There are provided in accordance with embodiments of the present invention a podiatric device and a method of using the same.

The podiatric device of the invention is useful for treating, preventing and alleviating the symptoms and discomfort caused by hallux valgus otherwise known as bunion deformity at the metatarsophalangeal joint of the hallux.

According to some embodiments of the invention, the podiatric device includes a foothold with a first hinge forming element and an anterior part with a second hinge forming element.

According to some embodiments of the invention, the anterior part of the podiatric device includes a structural element dedicated for engagement with at least one phalangeal appendage, i.e. a digit of the foot.

According to some embodiments of the invention, the podiatric device includes first and second hinge forming elements forming a hinge allowing angular translation of the anterior part relatively the foothold.

According to some embodiments of the invention, the podiatric device has first and second hinge forming elements forming a telescopic hinge allowing length translation of the anterior part relatively the foothold.

According to some embodiments of the invention the hinge includes a controllable pivoting movement.

### BRIEF DESCRIPTION OF THE DRAWINGS

The present invention will be understood and appreciated more fully from the following detailed description taken in conjunction with the appended drawings in which:

FIG. 1A is a bottom view of a foothold of an embodiment of a podiatric device of the present invention;

FIG. 1B is a side view of the foothold of the podiatric device of the present invention;

FIG. 1C is a top view of the foothold of the podiatric device of the present invention;

FIG. 2 is a top view of an anterior part of the podiatric device, in accordance with some embodiments of the present invention;

FIG. 3A is an isometric view of a cushion of the podiatric device, in accordance with some embodiments of the present invention;

FIG. 3B is a top view of the cushion of the podiatric device of the present invention;

FIG. 4 is a top view of the podiatric device, assembled, in accordance with some embodiments of the present invention;

FIG. 5 is a schematic top view of the podiatric device shown in FIG. 4 implemented on a foot;

FIGS. 6 to 6D respectively are a bottom view of right anterior part, an enlarged view thereof, a bottom view of left anterior part, isometric view of the right anterior part and an isometric view of the left anterior part of the podiatric device in accordance with some preferred embodiments of the present invention;

FIGS. 7A to 7C are bottom views of a right anterior part of the podiatric device in accordance with some embodiments of the present invention;

FIG. 7D is an isometric view of the right anterior part of the podiatric device of FIGS. 7A-7C; and

FIG. 8 is a top view of the podiatric device, assembled, in accordance with other embodiments of the present invention.

While the invention is susceptible to various modifications and alternative forms, specific embodiments thereof have been shown by way of example in the drawings. The components in the drawings are not necessarily to scale, emphasis instead being placed upon clearly illustrating the principles of the present invention.

### DETAILED DESCRIPTION OF THE INVENTION

Illustrative embodiments of the invention are described below. In the interest of clarity, not all features of an actual implementation are described in this specification. It should be appreciated that in the development of any such actual embodiment, numerous implementation-specific decisions must be made to achieve the developers' specific goals, such as compliance with technology or business-related constraints, which may vary from one implementation to another. Moreover, it will be appreciated that the effort of such a development might be complex and time-consuming, but would nevertheless be a routine undertaking for those of ordinary skill in the art having the benefit of this disclosure.

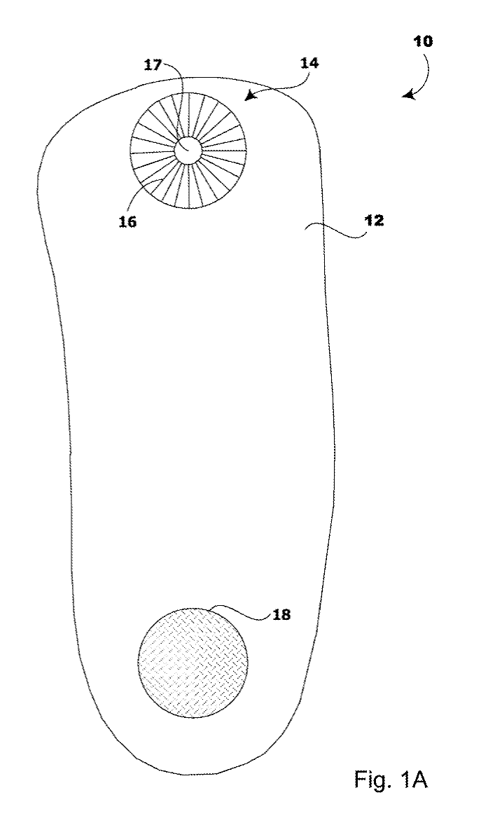

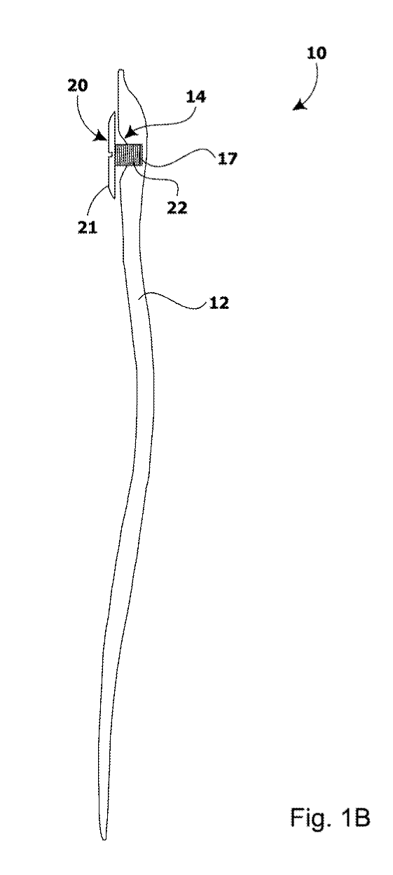

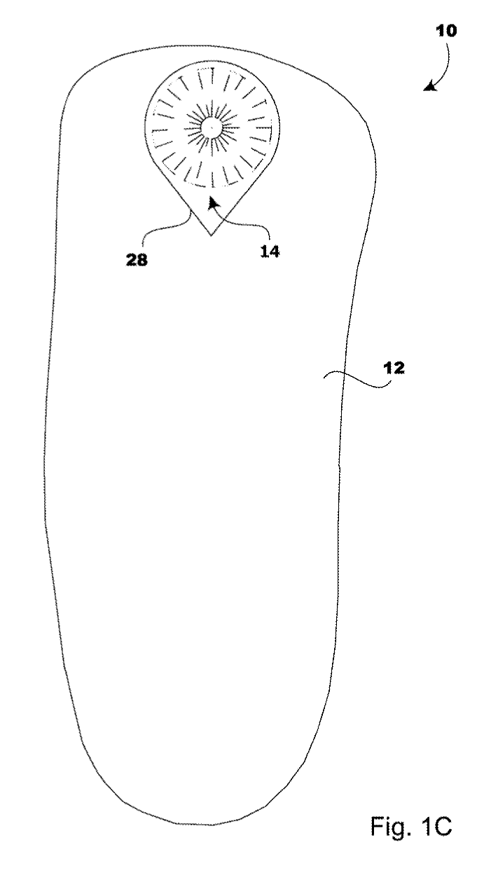

FIG. 1A shows a foothold 10 of the podiatric device, in accordance with some embodiments of the present invention. Foothold 10 comprises an abutment plate 12 having at the front portion thereof a concave or frusto-conical recess 14 furnished with a furrowed or corrugated surface 16 and an aperture 17 at the center thereof furnished with a screw threading. At the posterior portion, foothold 10 comprises a textured insert 18, used to facilitate an enhanced frictional force upon urging of the insert against the sole (not shown) thereby precluding a movement of the foothold relative to the sole.

Foothold 10 has an essentially planar conformation, a certain degree of pliability in the longitudinal direction and a degree of stiffness in a lateral direction. Thus, foothold 10 can be bent in the longitudinal direction so that textured insert 18 is brought somewhat closer to concave or frusto-conical recess 14, e.g. upwards/downwards from the plane of the page, but it is stiff and is not easily subjected to bending in the lateral direction, e.g. sidewise in the plane of the page, so that frusto-conical recess 14 remains essentially in a straight line with textured insert 18. The aforementioned pliability in the longitudinal direction and stiffness in the lateral direction can be achieved by forming foothold 10 from a sheet of plastic material that is relatively easily bent if a force applied in the directions facing away from the plane of the sheet, whereas exhibiting stiffness if a force applied in the directions paralleling the plane of the sheet. Foothold 10 is intended for use underneath human feet and hence the pliability in the longitudinal direction is required to allow walking and other activities requiring bending of the feet (not shown).

FIG. 1B illustrates that foothold 10 has a somewhat curved shaped side profile intended to ergonomically conform to the bottom surface of a human foot (not shown). Concave or frusto-conical recess 14 in foothold 10 comprises aperture 17 at the center thereof. Aperture 17 has a screw threading corresponding to accommodate bolt 20. Bolt 20 comprises a relatively flat and wide screw cap 21 and stud 22, having a screw threading corresponding to the screw threading of aperture 17.

FIG. 1C shows the top surface of abutment plate 12 of foothold 10 furnished with an insole lining (not shown) for the sake of comfort. Concave or frusto-conical recess 14 has a tear-shaped convex protrusion 28 on the top surface of abutment plate 12 and/or the insole lining, covering the lining. Tear-shaped convex protrusion 28 is intended to be accommodated at the metatarsal indent of the feet (not shown). It is noted that the insole lining may extend beyond the anterior edge of abutment plate 12, for the comfort of the phalangeal appendages, i.e. the digits of the foot, e.g. the toe.

FIG. 2 shows an anterior part 30 of the podiatric device comprising an essentially planar arm-element 32 having at the posterior portion thereof a convex or frusto-conical protrusion 34 has a furrowed or corrugated surface 36 and an aperture 37 at the center thereof.

Planar arm-element 32 has a certain degree of pliability in the longitudinal direction and a degree of stiffness in a lateral direction. Thus, element 32 can be bent in the longitudinal direction but is stiff and not easily bent in the lateral direction, similarly to abutment plate 12 of foothold 10.

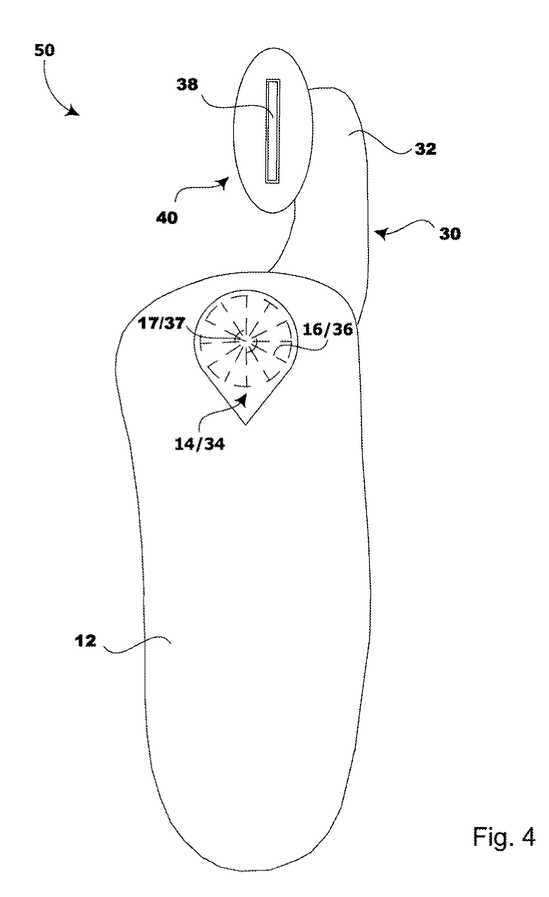

At the front portion of anterior part 30 there is a cushion holding strip 38, extending substantially perpendicularly to the planar surface of arm-element 32. Cushion holding strip 38 is used to install/hold a cushion 40 (FIG. 4) of the podiatric device, as will be explained in detail below. Convex or frusto-conical protrusion 34 is designed to conform to concave or frusto-conical recess 14 of foothold 10 shown FIG. 1A, so that the furrowed or corrugated surfaces 16 and 36 thereof interlock, thereby precluding a pivoting movement of anterior part 30 relatively to foothold 10 upon tightening of the anterior part to the foothold by bolt 20, shown in FIG. 1B.

It should be understood that the form of anterior part 30 comprising arm-element 32 is merely one example of a variety of possible forms therefor, beneficially implementable with the podiatric device of the invention. For example, arm-element 32 of anterior part 30 may be embodied in a plate-like element, continuously extending from the underneath of abutment plate 12 of the foothold 10 up to the front edge of the phalangeal appendages or up to the interior front edge of the shoe in which the podiatric device is accommodated, flanking a cushion holding member, such as cushion holding strip 38, on one side; thereby forming an abutment for all phalangeal appendages extending below the entire length thereof. Embodiments in which a plate-like anterior part is employed preferably combine the aforementioned insole lining, continuously extending beyond the anterior edge of abutment plate and covering the upper surface of the plate-like anterior part; thus conferring comfortability to the interaction of the phalangeal appendages therewith.

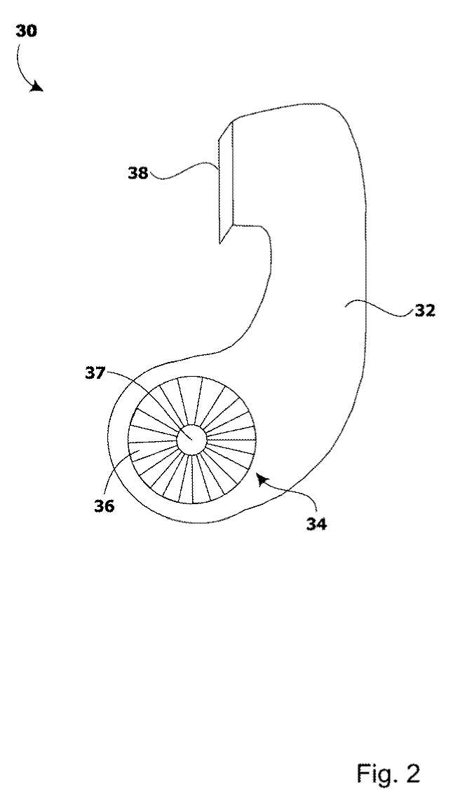

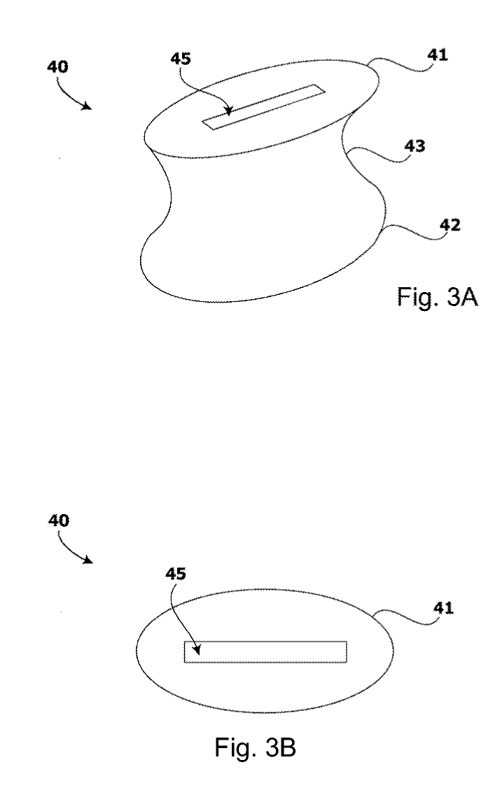

FIGS. 3A and 3B show an exemplary design of cushion 40 of the podiatric device. Cushion 40 has an elliptically-waisted curvilinear shape, wherein the top and the bottom elliptic circumferences 41 and 42, respectively, are somewhat larger than median elliptic circumference 43. Cushion 40 is preferably made of a material characterized by a relatively hard rubber-like consistency. Cushion 40 comprises a slot 45, adapted to receivingly accommodate cushion holding strip 38 of anterior part 30 FIG. 2. The insertion of cushion holding strip 38 of anterior part 30 into slot 45 of cushion 40 provides for a modular assembly of the podiatric device of the invention, so that a differently shaped cushion made from different materials can be used with the same cushion holding strip 38 of anterior part 30.

The elliptically-waisted curvilinear shape of cushion 40 is designed to conform in a sidewise manner to the hallux, (not shown), in a dedicated manner and exert a force thereon in the deflection away from the feet, so that median elliptic circumference 43 of cushion 40 is urged essentially against the most prominent portion of the hallux, whereas top and the bottom elliptic circumferences 41 and 42 are confining the hallux from above and below, respectively. It should be acknowledged, however, that the symmetrical elliptically-waisted shape of cushion 40 is a mere example of a vast array of shapes beneficially implementable with the podiatric device of the invention. Thus, cushion 40 can be laterally non-symmetrically shaped, so that the waisted profile on one side thereof differs from the waisted profile on the other side, thereby allowing for the cushion to be installed in a laterally-flipped orientation on the cushion holding strip 38 of anterior part 30, facing the waisted profile on the other side of the cushion vis-a-vis the hallux (not shown). In other instances, cushion 40 can have a waisted profile dedicated for engagement with the hallux on one side and be essentially flat on the other side, thereby providing for a more compact podiatric device of the invention that can be accommodated in standard shoes. The side of cushion 40 that is not intended for engagement with the hallux is preferably shaped to conform to the interior surface of the shoe. In yet another example, cushion 40 can be vertically non-symmetrically shaped, so that the top and the bottom elliptic circumferences thereof are unequal, thereby allowing for the cushion to be installed in a vertically-flipped orientation on cushion holding strip 38 of anterior part 30, disposing a larger/smaller elliptic circumference of the cushion vis-a-vis a upper/lower portion of the hallux (not shown).

FIG. 4 shows an assembled podiatric device 50, in accordance with some embodiments of the present invention. Podiatric device 50 is assembled from foothold 10, anterior part 30 and cushion 40. Convex or frusto-conical protrusion 34 at the posterior portion of anterior part 30 is appended to concave or frusto-conical recess 14 in abutment plate 12, so that the apertures 17 and 37 are coaxially aligned. Stud 22 of bolt 20 is then inserted throughout aperture 37 and further screwed into the screw threading of aperture 17, thereby providing the hinge 14, 34 with a controllable pivoting movement of anterior part 30 relative to abutment plate 12. Upon loosening bolt 20, an adjustment of the angular position of anterior part 30 relative to abutment plate 12 can be done; whereas upon tightening of bolt 20 furrowed or corrugated surfaces 16 and 36 are urged one to the other, thereby preventing pivoting movement whereby the angular position of anterior part 30 is fixed. Cushion 40 is installed onto cushion holding strip 38 of anterior part 30. In some embodiments, cushion 40 and anterior part 30 are not two distinct elements but rather a single part, wherein cushion holding strip 38 is adapted for a direct interaction with the hallux.

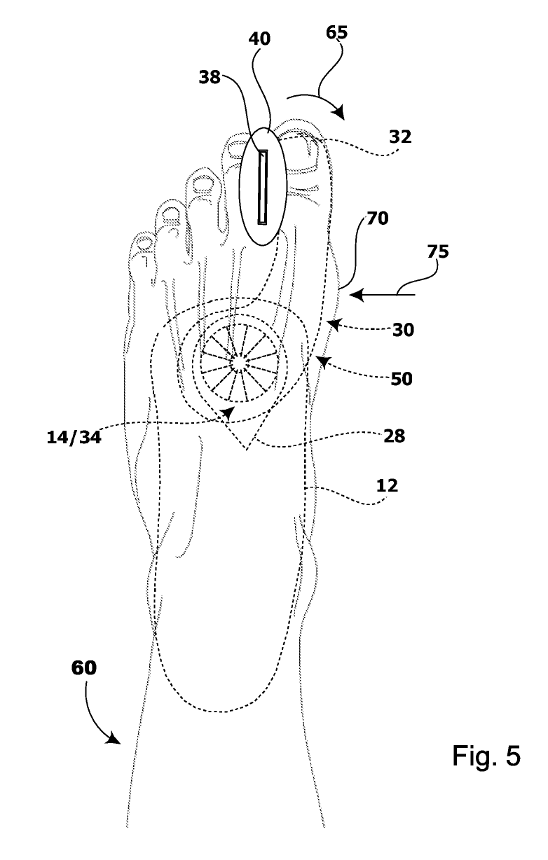

FIG. 5 shows podiatric device 50 implemented on foot 60; the features of the podiatric device that are obstructed by the foot are shown in dashed lines. Podiatric device 50 is disposed underneath foot 60, so that tear-shaped convex protrusion 28 formed on the top surface of abutment plate 12 above hinge 14, 34 is accommodated at the metatarsal indent of the foot, as shown. Anterior part 30 is frictionally engaged to foothold 10 at hinge 14, 34 with arm-element 32 in an appropriate angular position. Cushion 40 is inserted onto cushion holding strip 38 of anterior part 30 and engages the hallux of feet 60 in a dedicated manner and exerts a force thereon essentially in the direction of arrow 65. Consequently, cushion 40 forcefully spreads the hallux away from the foot, i.e. performs an abduction of the hallux of feet 60; whereby an adduction of deformity 70 at the metatarsophalangeal joint of the hallux, i.e. the hallux valgus or bunion of feet 60, essentially in the direction of arrow 75, is achieved. Optionally, anterior part 30 may be mounted directly underneath cushion holding strip 38 (FIG. 8).

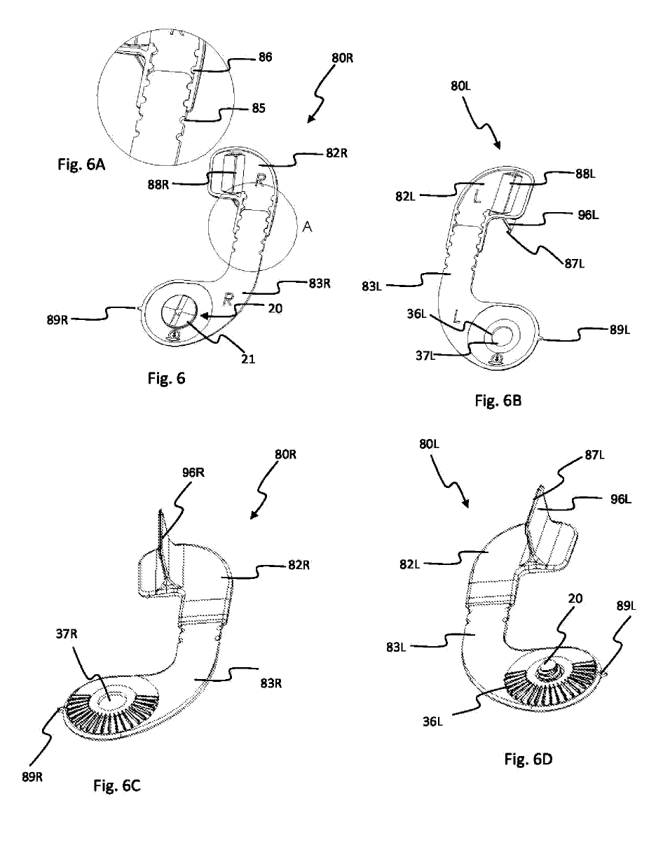

FIGS. 6-6D show embodiments wherein anterior part 30 of podiatric device 50 of the present invention is adapted to have the ability to assume a customized length. The anterior part 80 has a right anterior part 80R and a left anterior part 80L comprising right and left proximal sections 83R and 83L and right and left distal sections 82R and 82L, respectively. A proximal section 83 comprises notches 85, wherein distal section 82 comprises protrusions 86. Fitting protrusions 86 within notches 85 allows setting the relative distance between the cushion (not shown) and abutment plate (not shown).

A cushion holding strip 96L comprises a terminal portion 87L used to fixate the cushion 40. Cushion holding strip 96L further comprises a coupler 88L, which is used to fix cushion holding strip 96L within distal section 82L.

A pointing element 89L of proximal section 83L is used to indicate the angular position of anterior part 80L relatively to the abutment plate (not shown) by pointing onto appropriate indicia on the abutment plate. Proximal section 83L further comprises aperture 37 used to accommodate bolt 20. Bolt 20 comprise a relatively flat and wide cap screw 21 as elaborated above.

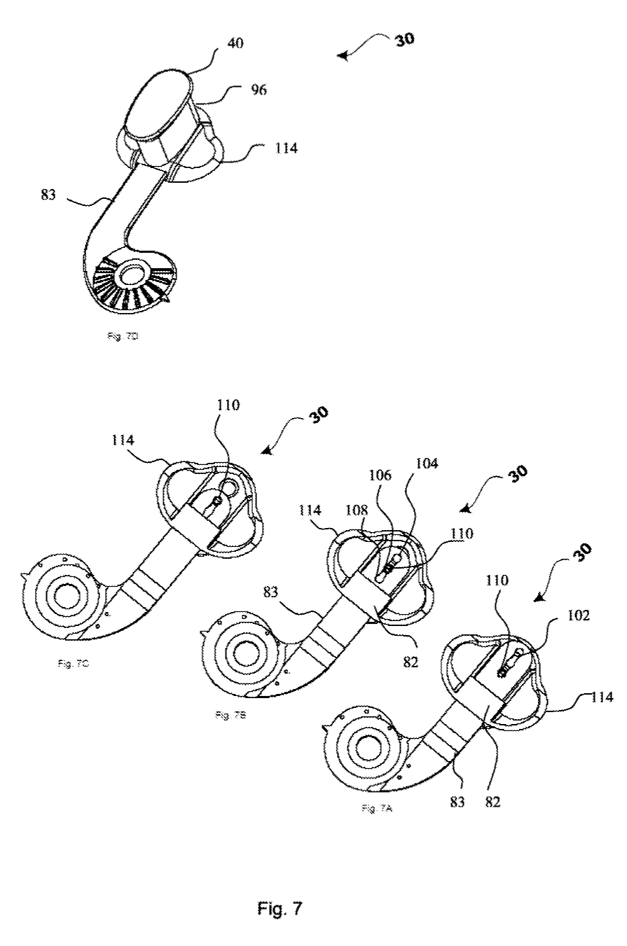

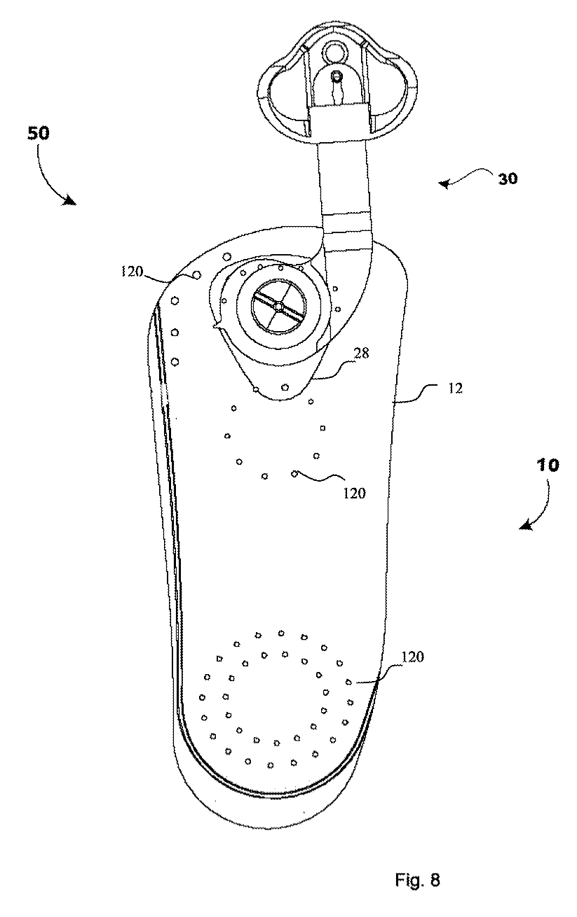

FIGS. 7A-7C show an additional embodiment for providing a customizable length to the anterior part 30 of podiatric device 50, in particular where the length of proximal section 83 can be adjusted via a length adjustment mechanism. In the length adjustment mechanism of this embodiment, proximal section 83 comprises a slit 102 and distal section 82 comprises a protrusion 110, corresponding to and insertable in the slit. Slit 102 has a plurality of spaced apart protrusion-receiving apertures, illustrated by three protrusion-receiving apertures 104, 106 and 108, for receiving protrusion 110. Alternatively, proximal section 83 comprises protrusion 110 and distal section 82 comprises slit 102. Such a construction allows protrusion 110 to be moved (e.g. slid along slit 102) to fit in any of the receiving apertures 104, 106 and 108 to thereby adjust and fix the length of proximal section 83 relative to abutment plate 12. Inserting protrusion 110 into slit 102 allows setting the distance between cushion 40 and abutment plate 12 (FIG. 8).

FIG. 7A shows anterior part 80 in its extended position, such that the longest length between the cushion 40 and abutment plate is produced. FIG. 7C shows anterior part 80 in its un-extended position, and FIG. 7B illustrates an intermediate position between the positions shown in FIGS. 7A and 7C. FIG. 7D shows a top view of anterior part 30 where one can see how cushion 40 is insertable into cushion holding strip 96 of anterior part 30. Optionally, distal section 82 further comprises a toe support 114, for adjacent toes.

FIG. 8 shows another embodiment of the invention wherein the foothold 10 is adapted to allow adjustment of the angle of anterior part 30 relative to podiatric device 50. Foothold 10 includes a plurality of friction elements such as nubs 120 on the surface of abutment plate 12. Nubs 120 are typically disposed at the heel area of abutment plate 12 and/or around the toe area and/or below tear-shaped convex protrusion 28, as shown. The friction elements (nubs 120) help hold podiatric device 50 in contact with footwear whereby the podiatric device can be more conveniently used with open shoes such as sandals, flip-flops, slingback shoes and the like.

It will be appreciated by persons skilled in the art that the present invention is not limited by what has been particularly shown and described herein above. Rather the scope of the invention is defined by the claims which follow:

What is claimed is:

1. A podiatric device for treating and/or preventing and/or alleviating symptoms and/or discomfort associated with hallux valgus, said device comprising:
a foothold, said foothold comprising:
an abutment plate, said abutment plate having a degree of pliability in the longitudinal direction and a predetermined degree of stiffness in a lateral direction;
a concave or frusto-conical surface at the anterior portion of said abutment plate; and
an aperture at the center of said concave or frusto-conical surface; and
an anterior part comprising:
an essentially planar element, said planar element having a degree of pliability in the longitudinal direction and a predetermined degree of stiffness in a lateral direction;
a concave or frusto-conical surface at the posterior portion of said planar element;
an aperture at the center of said concave or frusto-conical surface; and
a cushion holding strip, extending substantially perpendicularly to said essentially planar element; wherein said
concave/convex or frusto-conical surfaces respectively conform to each other, thereby forming a hinge that provides for an angular translation of said anterior part relative to said foothold, and said cushion holding strip is engageable with said hallux.

2. The device as in claim 1, further comprising a bolt, inserted through said aperture at the center of said concave or frusto-conical surface into said aperture at the center of said concave or frusto-conical surface, wherein the stud of said bolt and said aperture are furnished with respectively matching screw threading; thereby providing for tightening said anterior part relative to said foothold.

3. The device as in claim 2, wherein said bolt is adapted to be loosenable to allow adjustment of the angular position of said hinge and tightenable to fix the angular position of said anterior part relative to said abutment plate.

4. The device as in claim 1, wherein said concave/convex or frusto-conical surfaces comprise furrows or corrugation adapted to provide the hinge with a controllable pivoting movement of anterior part relative to the abutment plate.

5. The device as in claim 1, further comprising a cushion adapted to be mountable onto said cushion holding strip.

6. The device as in claim 5, wherein said cushion comprises a slot to accommodate said cushion holding strip.

7. The device as in claim 5, wherein said cushion comprises an elliptically-waisted curvilinear shape, wherein the top and the bottom elliptic circumferences thereof are larger than the median elliptic circumference.

8. The device as in claim 5, wherein said cushion has a profile adapted for engagement with the said hallux, at least on one side thereof.

9. The device as in claim 1, wherein said anterior part further comprises a length adjustment mechanism comprising a proximal section and a distal section, and said length adjustment mechanism is adapted to adjust the length of said proximal section relative to said abutment plate.

10. The device as in claim 1, wherein said device can be accommodated in standard shoes.

11. The device as in claim 1, wherein said foothold comprises a textured insert adapted to facilitate an enhanced frictional force with the sole.

12. The device as in claim 1, wherein said concave or frusto-conical recess forms a tear-shaped convex protrusion on the top surface of said abutment plate.

13. The device as in claim 12, wherein said tear-shaped convex protrusion is adapted for accommodation by the metatarsal indent of the foot.

14. The device as in claim 1, wherein said abutment plate comprises indicia to indicate the angular position of said anterior part relatively to said abutment plate.

15. A podiatric device associated with hallux valgus, said device comprising:
a foothold comprising an abutment plate and a first hinge forming element; and
an anterior part comprising an essentially planar element, a second hinge forming element and a structural element adapted for engagement with the valgus,
said device being characterized in that said first and second hinge forming elements form a hinge allowing angular translation of said anterior part relative to said foothold.

16. The device as in claim 15, wherein said hinge comprises a controllable pivoting movement.

17. The device as in claim 15, wherein said anterior part comprises proximal and distal sections.

18. The device as in claim 15, wherein said proximal section comprises notches, and wherein said distal section comprises respective cushion holding strip.

19. The device as in claim 16, wherein said cushion holding strip comprises a terminal portion used to fixate said cushion.

20. The device as in claim 16, wherein said cushion holding strip comprises coupler; used to modularly fixate said cushion holding strip within anterior part.
